(19) United States
(12) Patent Application Publication
(10) Pub. No.: US 2026/0326089 A1
(43) Pub. Date: Oct. 22, 2026

(54) VPO catalyst having improved selectivity and stability and process for production thereof

(71) Applicant: Clariant International Ltd, Muttenz (CH)

(72) Inventors: Sebastian BÖCKLEIN, Bruckmühl (DE); Gerhard MESTL, Bruckmühl (DE); Bernadette HOFMANN, Bruckmühl (DE); Gabriele BINDSEIL, Bruckmühl (DE); Anna ADLER, Bruckmühl (DE); Nina RAMMEL, Bruckmühl (DE)

(73) Assignee: Clariant International Ltd, Muttenz (CH)

(21) Appl. No.: 19/117,450

(22) PCT Filed: Oct. 19, 2023
(86) PCT No.: PCT/EP2023/079180

§ 371 Date: Apr. 1, 2025

(30) Foreign Application Priority Data
Oct. 21, 2022 (EP) 22202894.6

(51) Int. Cl. B01J 27/199; B01J 35/37; B01J 35/40; B01J 35/50; B01J 37/00; B01J 37/08; B01J 37/16; C07C 51/16

(52) U.S. Cl. CPC B01J 27/199; B01J 35/37; B01J 35/40; B01J 35/50; B01J 37/009; B01J 37/08; B01J 37/16; C07C 51/16; B01J 2523/27; B01J 2523/51; B01J 2523/55; B01J 2523/68

(57) ABSTRACT

The invention relates to a ZnO-containing VPO catalyst in the form of a shaped body for oxidation of hydrocarbons with molecular oxygen (e.g., of butane to maleic anhydride), wherein in transmission infrared spectroscopy, has a first absorption band having a maximum between 790 cm⁻¹ and 810 cm⁻¹ and possibly a second absorption band having a maximum between 820 cm⁻¹ and 840 cm⁻¹, wherein only the first absorption band is present or the intensity of the first absorption band is greater than the intensity of the second absorption band. The invention further relates to a process for producing a VPO catalyst of the invention, comprising the steps of: a) producing a catalyst precursor containing vanadyl hydrogenphosphate, b) forming the catalyst precursor to a shaped body, c) activating the shaped bodies to form a VPO phase, wherein after step a), ZnO is mixed with the catalyst precursor.

The invention relates to a VPO catalyst in the form of a shaped body for the oxidation of hydrocarbons with molecular oxygen, especially for the oxidation of butane to maleic anhydride with molecular oxygen, wherein the VPO catalyst contains between 0.05% by weight and 7.0% by weight of Zn which is partly present as ZnO and in transmission infrared spectroscopy has a first absorption band having a maximum between 790 cm⁻¹ and 810 cm⁻¹ and possibly a second absorption band having a maximum between 820 cm⁻¹ and 840 cm⁻¹, characterized in that only the first absorption band is present or the intensity of the first absorption band is greater than the intensity of the second absorption band.

The invention also relates to a process for producing a VPO catalyst of the invention, comprising the steps of:
a) preparing a catalyst precursor containing vanadyl hydrogen phosphate,
b) shaping the catalyst precursor to give shaped bodies,
c) activating the shaped bodies so as to form a VPO phase,
characterized in that ZnO is mixed with the catalyst precursor after step a).

The invention further relates to the use of a Zn compound in the form of a solid for stabilizing a VPO catalyst in particle form.

Maleic anhydride is a chemical intermediate of great economic importance. It is used for example in the production of alkyd and polyester resins, alone or else in combination with other acids. In addition, it is also a versatile intermediate for chemical synthesis, for example for the synthesis of γ-butyrolactone, tetrahydrofuran and butane-1,4-diol, which in turn are used as solvents or may be further processed to afford polymers, for example polytetrahydrofuran or polyvinylpyrrolidone.

Maleic anhydride is generally produced by partial oxidation of n-butane in the gas phase with molecular oxygen or with a gas containing molecular oxygen in the presence of a vanadium-phosphorus oxide catalyst (VPO catalyst) that contains vanadyl pyrophosphate (VPP). Vanadyl pyrophosphate in its pure form contains vanadium with a valency of +4 and is particularly suitable for the production of maleic anhydride from unbranched saturated or unsaturated hydrocarbons having at least four carbon atoms. Fixed-bed reactors and fluidized-bed reactors can both be used.

VPO catalysts have only low intrinsic activity in the reaction of n-butane to maleic anhydride. An adequate conversion therefore requires a large amount of catalyst. Moreover, VPO catalysts rank among the most costly non-noble metal catalysts of all, largely on account of the high cost of their starting materials. This sets the object of improving the catalyst performance (activity and selectivity) and also the lifetime and mechanical stability of such catalysts. It is known from the prior art that the performance of VPO catalysts can be improved by adding foreign elements to the vanadium-phosphorus oxide (VPO) phase, for example through the addition of molybdenum (Mo promoter or Mo doping).

U.S. Pat. No. 5,929,256 discloses the synthesis of an active vanadium-phosphorus catalyst modified with molybdenum for the production of maleic anhydride. In this synthesis, a compound substantially composed of pentavalent vanadium is reacted with a compound containing pentavalent phosphorus in an alcoholic medium suitable for the reduction of vanadium to an oxidation state less than 5. This results in the incorporation of molybdenum into the reaction product, with the formation of a solid precursor composition modified with molybdenum. The alcohol is removed, affording a dried, solid precursor composition modified with molybdenum. This is shaped into shaped bodies comprising the dried, solid precursor composition modified with molybdenum. The dried and shaped precursor compositions modified with molybdenum undergo activation to convert them into the active catalyst.

U.S. Pat. No. 5,070,060 discloses an improvement of the oxidation catalyst that is used for the partial oxidation of n-butane and that comprises mixed oxides of vanadium and phosphorus, zinc and lithium, comprising the addition of an agent for modifying the molybdenum compound in an amount of about 0.005 to 0.025/1 Mo/V to the catalyst during the reaction of the reduced vanadium compound with concentrated phosphoric acid. The addition of Mo generates a catalyst that is very stable, constitutes a more active system and is longer lasting than the unmodified catalyst.

U.S. Pat. No. 3,980,585 discloses a catalyst complex suitable for the conversion of normal C₄ hydrocarbons into maleic anhydride in the gas phase, comprising the components vanadium, phosphorus and copper and also one of the elements selected from Te, Zr, Ni, Ce, W, Pd, Ag, Mn, Cr, Zn, Mo, Re, Sm, La, Hf, Ta, Th, Co, U and Sn, preferably with an alkali metal or an alkaline earth metal.

U.S. Pat. No. 4,056,487 discloses one that is suitable for the partial oxidation of alkanes to the corresponding anhydrides, for example of normal C₄ hydrocarbons that are to be converted into maleic anhydride in the gas phase, comprising the components vanadium, phosphorus and oxygen, Nb, Cu, Mo, Ni, Co and Cr. Preference is given to components that additionally comprise one or more elements selected from Ce, Nd, Ba, Hf, U, Ru, Re, Li or Mg.

U.S. Pat. No. 4,515,904 discloses a process for producing a phosphorus-vanadium catalyst and a phosphorus-vanadium co-metal catalyst for use in the production of maleic anhydride from butane, the process comprising the reaction of a vanadium compound in an organic ether solvent having about 2 to about 10 carbon atoms with a phosphorus halide at a temperature of about 0° C. to about 200° C. in the presence of water or an aliphatic alcohol having about 1 to about 8 carbon atoms; removal of the solvent; and activation of the catalyst by addition of butane or another hydrocarbon starting product and of a phosphorus compound at an approximate temperature of about 300° C. to about 500° C.

U.S. Pat. No. 5,158,923 discloses an improvement of the oxidation catalyst that is used for the partial oxidation of n-butane and that comprises mixed oxides of vanadium and phosphorus, zinc and lithium, comprising the addition of an agent for modifying the molybdenum compound in an amount of about 0.005 to 0.025 Mo/V to the catalyst during the breakdown of the reduced vanadium compound by concentrated phosphoric acid. The addition of Mo generates a catalyst that is very stable, constitutes a more active system and is longer lasting than the unmodified catalyst.

U.S. Pat. No. 5,262,548 discloses an improvement of the oxidation catalyst that is used for the partial oxidation of n-butane and that comprises mixed oxides of vanadium and phosphorus, zinc and lithium, comprising the addition of an agent for modifying the molybdenum compound in an amount of about 0.005 to 0.025 Mo/V to the catalyst during the breakdown of the reduced vanadium compound by concentrated phosphoric acid. The addition of Mo generates a catalyst that constitutes a very stable, active system and has a longer lifetime than the unmodified catalyst.

WO 2013062919 A1 discloses a process for producing a promoted VPO catalyst, wherein the catalyst comprises the mixed oxides of vanadium and phosphorus and wherein the catalyst is promoted with at least one selected from niobium, cobalt, iron, zinc, molybdenum or titanium, the process comprising the steps of: (i) preparing a VPO catalyst comprising vanadyl pyrophosphate as principal component and comprising less than 5% by weight of vanadyl phosphate, (ii) contacting the VPO catalyst with a solution comprising as metal source a compound containing at least one metal selected from the group consisting of niobium, cobalt, iron, zinc, molybdenum or titanium so as form a metal-impregnated VPO catalyst, and (iii) drying the metal-impregnated VPO catalyst so as to form the promoted VPO catalyst. In one embodiment, a niobium-activated VPO catalyst is produced.

U.S. Pat. No. 5,280,003 discloses an improvement of an oxidation catalyst that is used for the partial oxidation of n-butane and that comprises mixed oxides of vanadium and phosphorus, zinc, lithium and molybdenum, in which production of the catalyst involves the performance of a crystallization step under static conditions that permit more uniform conditions for crystal growth. The static conditions are maintained by heating the solvent under reflux during crystallization.

U.S. Pat. No. 4,251,390 discloses the improvement of an oxidation catalyst used for the partial oxidation of n-butane that comprises vanadium and phosphorus mixed oxides, characterized in that, during the reaction of the reduced vanadium component with concentrated phosphoric acid, a zinc compound is added to the catalyst in an amount of 0.15 to 0.001 Zn/V. The addition of zinc generates a catalyst that is more readily activated and that is very stable to heating of the reaction system. Small amounts of lithium compounds and silicon compounds also have additional desirable catalytic effects without diminishing the benefit of the zinc compound.

DE 10 2014 004786 A1 relates to a catalyst comprising a vanadium-phosphorus oxide and an alkali metal in which the proportion by weight of the alkali metal in the vanadium-phosphorus oxide is within a range from 10 to 400 ppm based on the total weight of the vanadium-phosphorus oxide, to a process for the production thereof and to the use of the catalyst for the gas-phase oxidation of hydrocarbons, especially for the production of maleic anhydride.

In order to produce VPO catalysts that contain a VPP phase, it is usual to carry out a reduction of vanadium pentoxide (V₂O₅) in the simultaneous presence of phosphoric acid in an organic alcoholic solvent with benzyl alcohol as reductant, resulting in the formation alongside benzaldehyde of vanadyl hydrogen phosphate (VHP). The redox reaction (“reduction”) that proceeds here, in which vanadium in oxidation state V (V(V)) reacts to form the VHP phase, in which vanadyl species (VO²⁺) with vanadium in oxidation state IV (V(IV)) are present, is as follows:

V₂O₅+2H₃PO₄+Ph-CH₂—OH→2VOHPO₄*½H₂O+Ph-CHO+2H₂O (1)

In a subsequent activation step, the VHP phase is converted by the action of heat into the vanadyl pyrophosphate phase alongside the elimination of water.

2VOHPO₄*½H₂O→(VO)₂P₂O₇+1½H₂O (2)

A problem in the catalytic reaction of butane to maleic anhydride using VPO catalysts is that the process must be operated in a regime that is limited by pore diffusion. The porosity thus has a direct influence on the catalytic yield here. Care must therefore be taken to ensure that the pore structure is not adversely influenced during shaping (for example by tabletting). This does however have the consequence that the mechanical stability of the shaped bodies suffers. This must nevertheless be high enough for the tablets to survive the filling process (drop into a reaction tube approx. 6 m in length) intact. Otherwise, the smaller fragments would result in the dynamic pressure in the process being too high, leading to high compressor costs or reduced throughputs. A technical object is thus to increase the mechanical strength of the shaped bodies without adversely affecting the pore structure. At the same time, there is a need for VPO catalysts having improved performance, i.e. improved activity, selectivity and stability.

The object of the present invention was therefore to provide an improved VPO catalyst for the gas-phase oxidation of hydrocarbons, especially for the production of maleic anhydride, that has increased catalytic performance, in particular an improved selectivity, compared to catalysts commonly used up to now and at the same time has substantially improved mechanical strength.

The object is achieved by a VPO catalyst in the form of a shaped body for the oxidation of hydrocarbons with molecular oxygen, especially for the oxidation of butane to maleic anhydride with molecular oxygen, wherein the VPO catalyst contains between 0.05% by weight and 7.0% by weight of Zn which is partly present as ZnO and in transmission infrared spectroscopy has a first absorption band having a maximum between 790 cm⁻¹ and 810 cm⁻¹ and possibly a second absorption band having a maximum between 820 cm⁻¹ and 840 cm⁻¹, characterized in that only the first absorption band is present or the intensity of the first absorption band is greater than the intensity of the second absorption band.

The intensity of the absorption band is determined here by in each case drawing a first straight line such that it makes tangential contact with the spectrum immediately to the left and right beneath the band in question, then running a second straight line proceeding from the maximum of the band in the direction of constant wavelength and decreasing absorption and determining the point of intersection of the two straight lines, where the intensity of the absorption band is defined as the length between maximum and point of intersection. The ratio of the intensity of the first absorption band to the intensity of the second absorption band here is preferably more than 1.1, preferably more than 2.

The shaped bodies according to the invention have a side crush strength of more than 25 N, preferably between 25 N to 200 N, the side crush strength having been determined with a Zwick Z0.5 tester using the standard ASTM D4179 at a constant force rate of 20.0 N/s, wherein 100 tablets were each positioned individually and measured with the cylinder axis parallel to the measuring jaw surface so as to determine an average breaking force, which represents the side crush strength.

When analysed by powder x-ray diffractometry using Cu-Kα radiation, the VPO catalyst according to the invention shows reflections at 31.7 to 31.9°, 34.3 to 34.5° and 36.2° to 36.4°.

The VPO catalyst according to the invention includes the VPO phase or comprises or consists essentially thereof. For example, the VPO catalyst according to the invention includes the VPO phase in a content of more than 70% by weight, preferably more than 80% by weight, particularly preferably more than 90% by weight, based on the total weight of the VPO catalyst. In addition, the VPO catalyst may include VPP, dopings and unreacted oxides of the starting materials, for example vanadium pentoxide or phosphorus oxide.

The VPO catalyst according to the invention may optionally contain between 0.1% by weight and 1% by weight, preferably between 0.4% by weight and 0.7% by weight, of Mo, based on the total weight of the VPO catalyst.

The VPO catalyst according to the invention may however also contain alkali metals such as Na and/or K, preferably 80 to 300 ppm of alkali metal. In addition, the VPO catalyst according to the invention may also contain carbon, for example in the form of graphite, for example in an amount of from 3% by weight to 5% by weight based on the total weight of the catalyst. The graphite present serves for example as a tabletting aid.

The VPO catalyst according to the invention includes ZnO, but may also contain further Zn compounds. The content of Zn in the VPO catalyst arising through the presence of ZnO and possibly present further Zn compounds must be between 0.05% by weight and 7.0% by weight, preferably between 0.1% by weight and 6.0% by weight, more preferably between 0.2% by weight and 4.0% by weight, most preferably between 0.7% by weight and 3.0% by weight, of Zn, in each case based on the total weight of the catalyst.

The VPO catalyst preferably has the following elemental composition:
0.05% by weight to 7% by weight of Zn,
0% by weight to 0.7% by weight of Mo,
26% by weight to 31% by weight of V,
17% by weight to 21% by weight of P,
3% by weight to 5% by weight of C,
the rest being oxygen, in each case based on the total weight of the VPO catalyst.

The VPO catalyst according to the invention comprises ZnO and—provided the content of these two phases is sufficiently high—the catalyst exhibits in an XRD powder diffractogram recorded using Cu-Kα radiation reflections typical of the ZnO phase. In particular, sharp reflections at 31.7° to 31.9°, 34.3° to 34.5°, 36.2° to 36.4° are detected.

The presence of ZnO has the effect according to the invention of stabilizing the catalyst particles so that they have a higher mechanical strength compared to the strength when ZnO is absent. However, the presence of ZnO also has the effect of improving the catalytic performance (i.e. the activity, selectivity and stability) of the catalyst particles, in particular increasing the selectivity.

The VPO catalyst according to the invention is present in the form of shaped bodies; the shape of the shaped bodies can be of varying design, depending on the desired contact time, flow rate and dynamic pressure during the catalytic reaction. The VPO catalyst in the form of a shaped body is understood as meaning shaped bodies produced by a shaping step such as tabletting. When present in large quantity in a tube-bundle reactor, the shaped bodies according to the invention form a layer or a catalyst bed through which the reactants butane, especially n-butane, and air are passed.

According to the invention, the shaped body should not be smaller than a minimum size that corresponds for example to the size whereby the shaped body is unable to fit into an imaginary cube of 3 mm×3 mm×3 mm without exceeding its limits at any point.

For example, the shaped bodies according to the invention may be in the form of a customary cylindrical shape. The cylinder in that case has a height (length along the cylinder axis) of 3 mm to 8 mm and an essentially round base surface with a diameter of 3 mm to 8 mm. Preferably, the cylinder has a central axial opening; this can have for example a diameter of 1 mm to 3 mm.

For example, the shaped body according to the invention may have a height of 4.7 mm, an external diameter of 4.7 mm and a central axial opening with a diameter of 1.3 mm. This shaped body then has a geometric surface area of 1.2 cm², a volume of 0.075 cm³ and a mass of 0.12 g. Filling a large quantity of such shaped bodies into a 21 mm reactor results in a poured density of 0.85 g/cm³ to 0.89 g/cm³.

For example, the shaped bodies according to the invention may have a height of 5.6 mm, an external diameter of 5.5 mm and a central axial opening with a diameter of 2.3 mm. These shaped bodies then have a geometric surface area of 1.77 cm², a volume of 0.111 cm³ and a mass of 0.18 g. Filling a large quantity of such shaped bodies into a 21 mm reactor results in a poured density of 0.72 g/cm³ to 0.76 g/cm³.

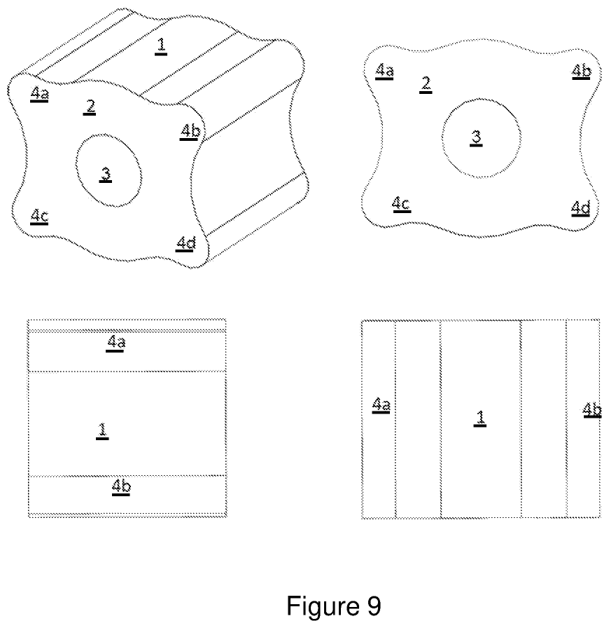

Preferred shaped bodies for use in the reactor concept according to the invention are those described in EP 2643086 A1. The preferred double-alpha shape is especially characterized in that each individual shaped body is in each case designed in the form of a cylinder having an outer base surface [1], a cylinder surface [2], a cylinder axis and at least one continuous opening [3] running parallel to the cylinder axis, and the outer base surface [1] of the cylinder contains at least four lobes [4a, 4b, 4c, 4d], wherein a geometric base body enclosing the shaped bodies is a prism having a prism base surface having a length and a width, wherein the length is greater than the width and wherein the lobes [4a, 4b, 4c, 4d] are enclosed by prism corners of the prism base surface (FIG. 9).

Preference is given to a shaped body in a double-alpha shape with a height (length along the cylinder axis) of 3 mm to 8 mm, a length of 5 mm to 9 mm and a width of 4 mm to 8 mm and an internal hole diameter of 1 mm to 4 mm. Preference is for example given to a shaped body in a double-alpha shape with a height of 5.6 mm, a length of 6.7 mm, a width of 5.8 mm and an internal hole diameter of 2.1 mm. These shaped catalyst bodies have a geometric surface area of 2.37 cm², a volume of 0.154 cm³ and a mass of 0.24 g. Filling into a 21 mm reactor results in a poured density of 0.60 g/cm³ to 0.62 g/cm³.

The VPO catalyst/shaped body of the invention has a side crush strength of more than 25 N, preferably between 25 N to 200 N. Particularly preferably, the VPO catalyst/shaped body has a side crush strength of more than 30 N to 150 N, even more preferably of more than 35 N to 100 N.

The VPO catalyst/shaped body of the invention in a cylinder shape has a side crush strength of more than 25 N, preferably between 25 N to 50 N. Particularly preferably, the VPO catalyst/shaped body has a side crush strength of more than 30 N to 45 N, even more preferably of more than 35 N to 40 N.

The VPO catalyst/shaped body of the invention in a double-alpha shape has a side crush strength of more than 50 N, preferably between 50 N to 200 N. Particularly preferably, the VPO catalyst/shaped body has a side crush strength of more than 100 N to 170 N, even more preferably of more than 120 N to 150 N.

The invention further relates to a process for producing a VPO catalyst of the invention, comprising the steps of:
a) preparing a catalyst precursor containing vanadyl hydrogen phosphate,
b) shaping the catalyst precursor to give shaped bodies,
c) activating the shaped bodies so as to form a VPO phase,
characterized in that ZnO is mixed with the catalyst precursor after step a).

In a process step a), a catalyst precursor containing vanadyl hydrogen phosphate is prepared in a generally known manner. Typically, in this case, in a reaction mixture, a V(V) compound in solution is reduced in a reduction step with the aid of a reductant in the presence of a P(V) compound, optionally a Mo compound. For example, the reaction mixture may consist of 45% by weight to 90% by weight of solvent, 5% by weight to 15% by weight of reductant, 5% by weight to 15% by weight of a V(V) compound, up to 1% by weight of a Mo compound and between 5% by weight to 25% by weight of a P(V) compound. More specifically, 60% by weight to 70% by weight of isobutanol, 5% by weight to 15% by weight of benzyl alcohol, 5% by weight to 15% by weight of vanadium pentoxide, 0.05% by weight to 0.2% by weight of (NH₄)₂Mo₂O₇ and 10% by weight to 20% by weight of phosphoric acid, in each case based on the total weight of the reaction mixture, may for example be initially charged as the reaction mixture.

The V(V) compound used as the starting material in the reaction mixture for producing the vanadyl hydrogen phosphate is a compound containing vanadium in oxidation state V and is preferably V₂O₅. The P(V) compound used as the starting material in the reaction mixture for producing the vanadyl hydrogen phosphate is a compound containing phosphorus in oxidation state V and is preferably phosphoric acid, or a phosphate salt such as Na₃PO₄. If phosphoric acid (H₃PO₄) is used, it is preferably anhydrous (100% phosphoric acid) or phosphoric acid containing only small amounts of water, i.e. phosphoric acid having a concentration of 98 to 100%, preferably 99 to 100% (percent values refer to the percent content by weight of pure phosphoric acid in relation to the weight of the water-phosphoric acid mixture, as customarily expressed). Alternatively, for the preparation of the reaction mixture for producing the vanadyl hydrogen phosphate it is possible to use phosphoric acid having a concentration of more than 100%, which reacts immediately with any water initially present in the reaction mixture, with the formation of phosphoric acid having a concentration of 98 to 100%, preferably 99 to 100%, more preferably 100%, with the result that the reaction mixture does not contain phosphoric acid having a concentration of more than 100% and at the same time not more than 0.2% by weight of water, based on the weight of the reaction mixture, remains in the reaction mixture.

The molybdenum compound that may optionally be used as starting material in the reaction mixture for producing the vanadyl hydrogen phosphate is any desired compound that contains molybdenum, for example molybdenum trioxide, ammonium heptamolybdate ((NH₄)₆Mo₇O₂₄)*4H₂O), ammonium paramolybdate ((NH₄)₆Mo₇O₂*4H₂O), meta molybdate, molybdic acid (H₂MoO₄) and salts thereof, such as (NH₄)₂MoO₄, Na₂MoO₄, K₂MoO₄ or (NH₄)₂Mo₂O₇.

The reductant present in the reaction mixture for producing the vanadyl hydrogen phosphate may be any desired reductant capable of reducing the V(V) compound such that vanadyl hydrogen phosphate forms at least in part. Preferably, the reductant is an organic reductant such as ethanol, isobutanol or an aromatic alcohol, including in particular benzyl alcohol.

The solvent present in the reaction mixture for producing the vanadyl hydrogen phosphate is preferably an alcohol, more preferably a high-boiling aliphatic alcohol, especially isobutanol, alternatively ethanol or isopropanol.

The starting materials are provided in an appropriate reaction vessel in order to perform the reduction step and in order to obtain the vanadyl hydrogen phosphate of the catalyst precursor. The reaction mixture is in this case heated to above room temperature, for example up to a temperature of 100° C. Since the reduction is preferably carried out with stirring in a reflux step, the reaction vessel preferably has a reflux condenser and a device for stirring the reaction mixture. Optionally, the reaction vessel is equipped with a device that permits the removal from the reaction mixture of the water formed during the reduction, i.e. a water separator, for example a Dean-Stark trap.

The reduction to give vanadyl hydrogen phosphate is effected preferably under reflux at standard pressure, the temperature being increased here in line with the boiling point of the solvent used; it is preferable that the process according to the invention involves the performance of just a single reflux step. The catalyst precursor preferably contains vanadyl hydrogen phosphate as the main phase or can even consist essentially of a vanadyl hydrogen phosphate phase. The molybdenum possibly likewise present in the catalyst precursor may be present in the form of doping of the vanadyl hydrogen phosphate phase, molybdenum doping being understood as meaning that the molybdenum is either incorporated into the vanadyl hydrogen phosphate phase or is present on the surface thereof. In addition to the vanadyl hydrogen phosphate phase, it is however possible for the reduction to result in the formation of further vanadium-phosphorus mixed oxides in which vanadium has an oxidation state of IV or even Ill. The reduction does not need to proceed to completion, which means that proportions of the V(V) compound and of the P(V) compound also remain in the catalyst precursor. In the catalyst precursor, vanadium is however typically present in an average oxidation state of 3.8 to 4.2.

The water formed during the reduction may be removed from the reaction mixture during the reduction. The removal of water during the reduction is in the prior art effected either physically, for example with the aid of a water separator, or chemically through the use of compounds that bind the water, for example drying agents or anhydrides such as phosphoric acid having a concentration of more than 100%. The reaction mixture may comprise, for example, anhydrides that react with and bind water, in particular, the reaction mixture may comprise phosphoric acid having a concentration of more than 100%.

The suspension obtained after the reduction step may be filtered for example in an atmosphere of an inert gas such as nitrogen or a noble gas. In this context, inert gas means any gas that does not react with the catalyst precursor under the specified conditions during the filtration, but at the same time displaces oxygen from the air so as to minimize the risk of an explosion. The filtration is carried out by means known to those skilled in the art, typically by means of a filter press, a decanter or through a filter funnel. The filtration affords an uncalcined catalyst precursor, which is still wet with solvent.

The catalyst precursor obtained by the filtration (the solid filtration residue) may subsequently be dried; this is effected typically at a temperature above room temperature, for example at a temperature of up to 150° C. under reduced pressure/vacuum or under inert gas, so as to obtain a dried catalyst precursor. In this context, inert gas means any gas that does not react with the catalyst precursor under the conditions of the drying, but at the same time displaces oxygen from the air so as to minimize the risk of an explosion, for example nitrogen or a noble gas. Preferably drying proceeds under reduced pressure/vacuum between 50° C. and 150° C., preferably between 90° C. and 140° C.

Alternatively or optionally, drying can be followed by calcining so as to obtain the catalyst precursor. The calcining is effected at elevated temperature between 150° C. and 350° C., preferably between 230° C. and 290° C., under inert gas. In this context, inert gas means any gas that does not react with the catalyst precursor under the conditions of the calcining, but at the same time displaces oxygen from the air so as to minimize the risk of an explosion, for example nitrogen or a noble gas.

Optionally, graphite may be added to the dried catalyst precursor so as to facilitate the shaping in process step b). The dried catalyst precursor may also undergo a compaction and/or granulation so as to obtain a compacted or granulated dried catalyst precursor. In this case, the catalyst precursor can be compacted into plates with a roller compactor at a compaction pressure of 190 bar, a gap width of 0.60 mm and a roller speed of 7 rpm and be granulated through a 1 mm screen.

In process step b), the catalyst precursor obtained is shaped into shaped catalyst bodies; this can be effected for example through tabletting. This is accomplished for example by pressing the granulate with a rotary tablet press into the desired tablet shape having the appropriate height.

In the subsequent process step c), the shaped catalyst bodies obtained undergo activation at a temperature above 200° C. The activation is effected typically in a gas mixture consisting of air, inert gas and water vapour, wherein the inert gas used may be any gas that does not react with the shaped catalyst body under the specified conditions during the activation and is particularly preferably nitrogen or a noble gas. Alternatively, the activation may also be effected in process gas, i.e. in a gas mixture containing air and butane. The activation is effected at a temperature within a range from 300° C. to 500° C., preferably within a range from 350° C. to 450° C. Activation affords the finished VPO catalyst as the product of the process. The tableted VPO catalyst typically has a side crush strength of above 25 N, typically between 25 N to 200 N.

The process according to the invention for producing the VPO catalyst in the form of shaped bodies is characterized in that ZnO is added to the catalyst precursor or mixed with it after process step a). The ZnO, i.e. (zinc(II) oxide), is used here as powder mediator solid. The addition of the ZnO to the catalyst precursor can also be effected with the aid of a metering device and a mixer. In order to produce the catalyst of the invention, sufficient ZnO is generally added. For example, 0.5% by weight to 7.0% by weight, preferably 1.0% by weight to 5.0% by weight, more preferably 1.5% by weight to 3.5% by weight, of ZnO in solid form, based on the weight of the dried catalyst precursor (without ZnO), may be added.

The ZnO is added to the catalyst precursor, i.e. after production thereof in process step a), for example by a reduction step, but before the shaping of the shaped catalyst bodies in process step c). Preferably, the ZnO is added to the dried catalyst precursor, more preferably to the dried and calcined catalyst precursor.

In a further preferred embodiment, as well as the ZnO, a further solid metal compound, preferably a metal compound in powder form, is added. It is preferable that a solid Mg compound is also present in the binder, for example MgO.

The invention relates also to the use of ZnO for stabilizing a VPO catalyst in the form of a shaped body and to the use of ZnO as binder for a VPO catalyst in the form of a shaped body.

The invention further relates to a process for producing maleic anhydride by catalytic oxidation of n-butane, wherein a reactant gas comprising oxygen and n-butane is passed through a reactor tube containing a bed of the VPO catalysts of the invention.

The bed of the VPO catalysts in the reactor tube consists of the VPO catalysts/shaped bodies of the invention, which are poured into the reactor tubes and, after resting, afford a catalyst bed. The reactor tube is preferably part of a large number of reactor tubes of a tube-bundle reactor, such as ones for the industrial production of maleic anhydride known to those skilled in the art. During the reaction, the bed of the VPO catalysts is present at a temperature of between 300° C. and 420° C. The reactant gas may contain for example between 0.2% to 10% by volume of n-butane and between 5% and 50% by volume of oxygen and is passed through the reactor tube at a space velocity of 1100 h⁻¹ to 2500 h⁻¹, preferably 1300 h⁻¹ to 2000 h⁻¹.

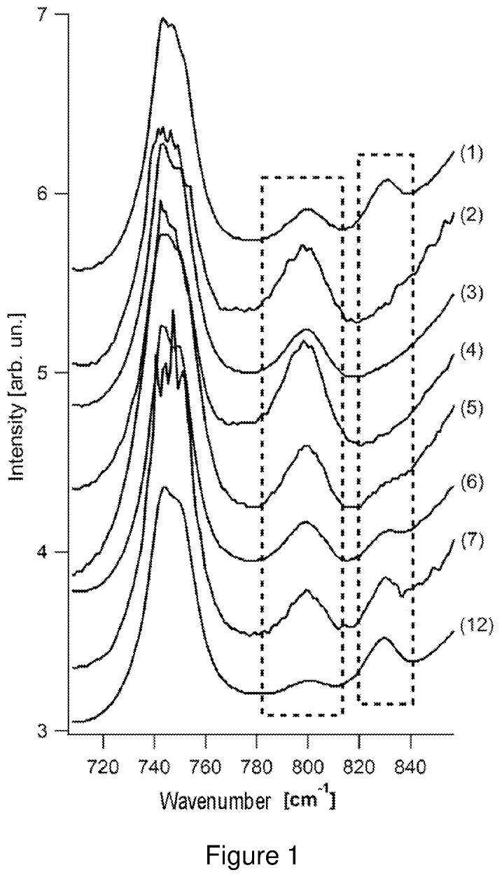

FIG. 1: IR spectra of the samples according to examples 1 to 7 and example 12.

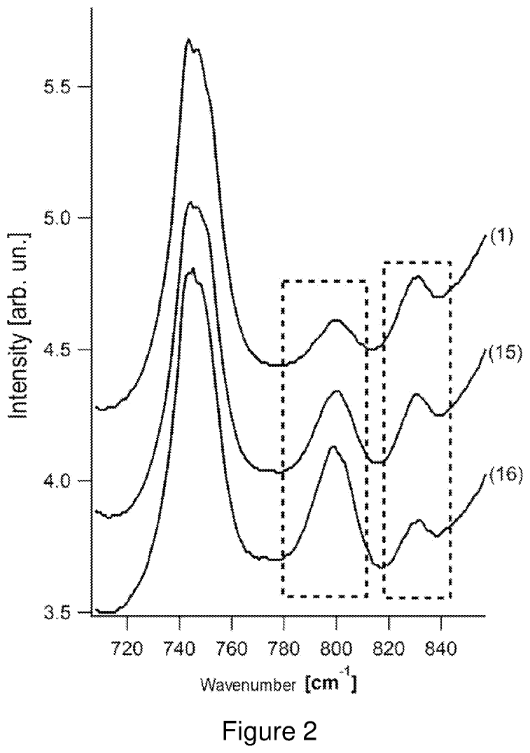

FIG. 2: IR spectra of the samples according to examples 1 and 15 and 16.

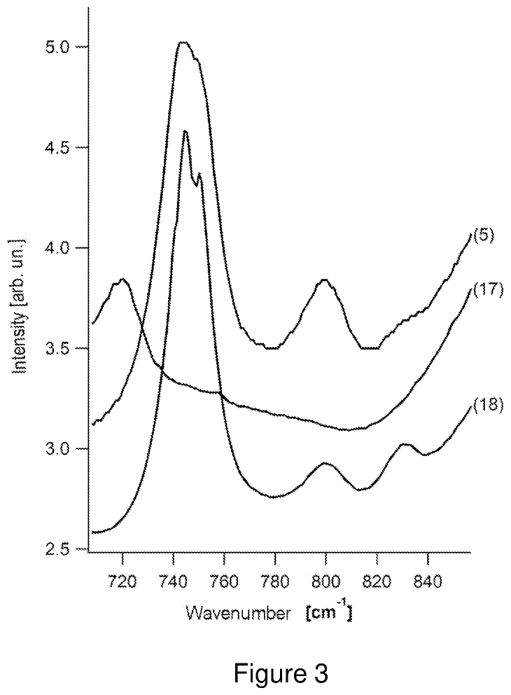

FIG. 3: IR spectra of the samples according to example 5 and comparative examples 17 and 18.

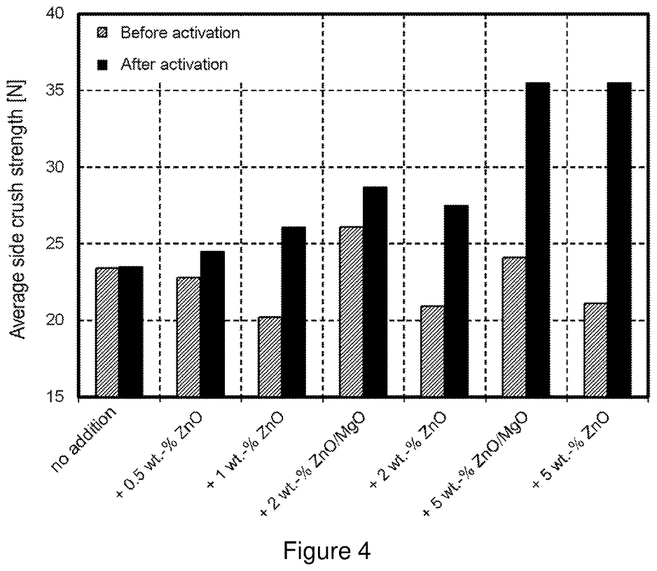

FIG. 4: Influence of the use of ZnO and ZnO/MgO solids in the synthesis on the stability of the VPO catalysts according to examples 1 to 7.

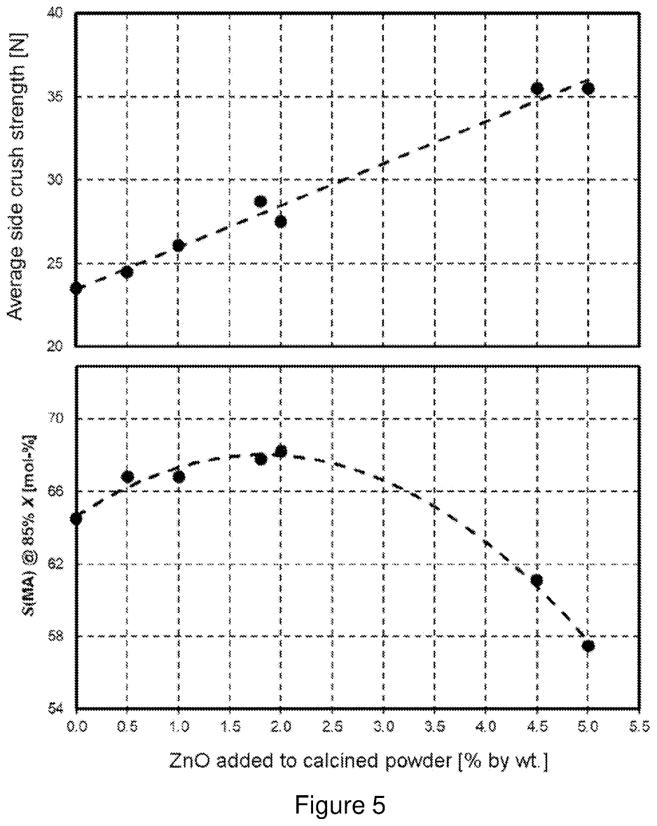

FIG. 5: Change in the side crush strength and selectivity of the VPO catalysts with varying amounts of added Zn.

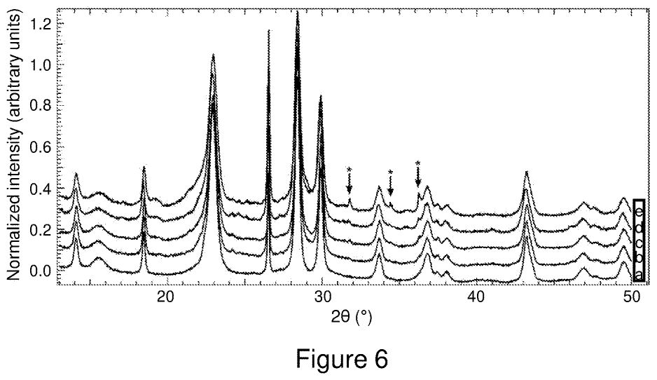

FIG. 6: XRD diffractograms of the VPO catalysts according to examples 1 to 5.

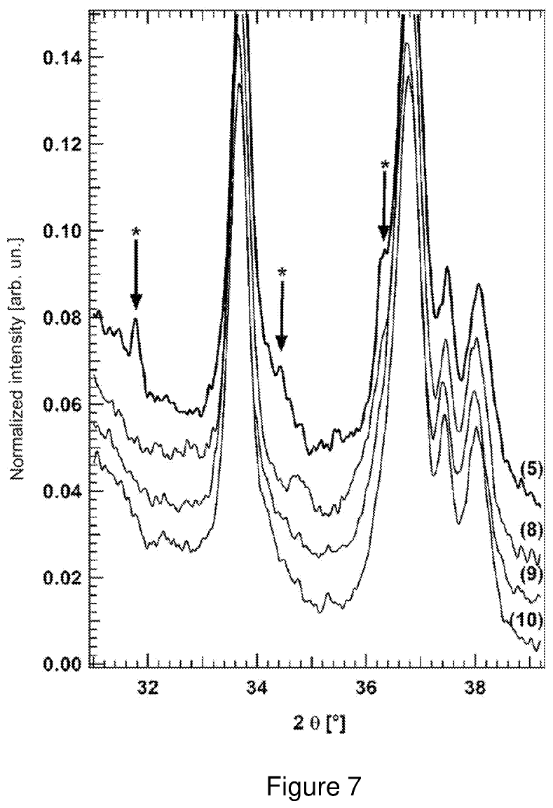

FIG. 7: XRD diffractograms of the VPO catalysts according to examples 5 and 8 to 10.

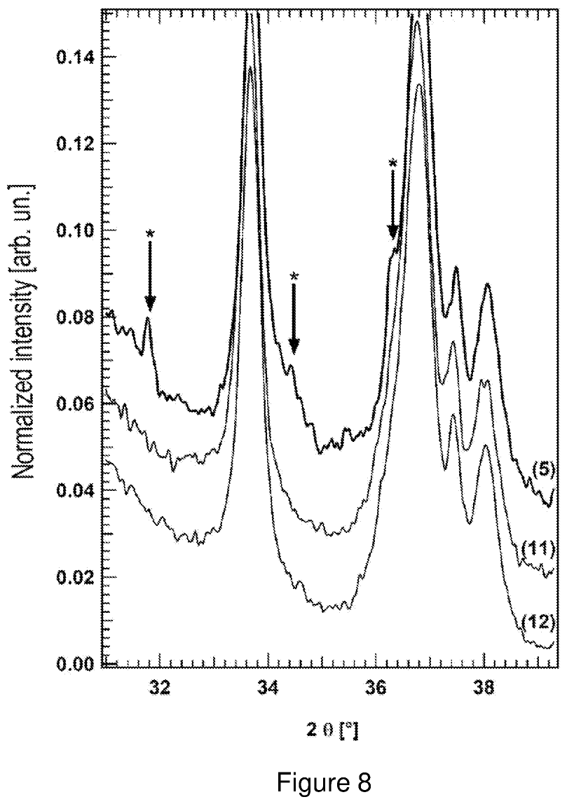

FIG. 8: XRD diffractograms of the VPO catalysts according to examples 11 and 12 compared to that of the VPO catalyst produced according to the invention as per example 5.

FIG. 9: Representations of the preferred catalyst particle, the “double-alpha shape” from four different perspectives.

The numbers in brackets in the figures refer in each case to the sample prepared by the corresponding example.

### EXAMPLES

### Example 1 (Comparative)

### Apparatus Used

A heating mantle, inside which is a 2 L four-necked flask, is positioned on a laboratory jack. Present in the central neck of the four-necked flask is a half-moon impeller with corresponding stirrer seal that is connected to the stirrer unit by means of a stirrer coupling. Present in the right-hand neck is a thermometer and in the left-hand neck a riser to a reflux condenser. The frontally positioned central neck is used for filling with the chemicals, after which the nitrogen inlet is connected there. The entire apparatus can also be flushed with nitrogen. The nitrogen for this is first passed through a gas-wash bottle and then into the apparatus and passes out at the top of the condenser, again through a gas-wash bottle.

### Preparation of the Catalyst Precursor

1069.5 g of isobutanol and 156.0 g of benzyl alcohol are first added. 150 g of V₂O₅ is added with stirring. The V₂O₅ addition is followed by addition of 2.52 g of ammonium dimolybdate. 232.50 g of phosphoric acid (100%, anhydrous) is then added to the suspension and the mixture is heated under reflux under N₂ for 10 h.

### Filtration

After cooling the suspension containing the catalyst precursor product in solid form, the suspension is transferred from the four-necked flask to a filter funnel and the liquid is removed by suction. The damp filter cake is pressed dry overnight in a press at 14 to 18 bar.

### Drying/Calcining

The pressed filter cake is transferred to the evaporator flask of a rotary evaporator. The filter cake is dried at 110° C. overnight under water-jet vacuum. The powder dried in this way is placed in a furnace in a suitable calcining pot and calcined in an N₂ atmosphere at temperatures of 200° C. to 300° C. for 9 h.

### Compacting/Tabletting

Before compacting/tabletting, 5% by weight of graphite is added to the calcined pulverulent catalyst precursor product and this is mixed homogeneously using a drum hoop mixer. This powder is compacted into plates with a roller compactor at a compaction pressure of 190 bar, a gap width of 0.60 mm and a roller speed of 7 rpm and granulated through a 1 mm screen.

The granulate is pressed with a rotary tablet press into the desired tablet shape having the appropriate height, for example 5.6×5.6×2.3 mm, and side crush strength.

### Activation to Pyrophosphate

The activation to form vanadium pyrophosphate is carried out under controlled conditions in a retort installed in a programmable furnace. The calcined tablets are evenly filled into the retort and the retort is sealed tightly. The catalyst is then activated in a moist air/nitrogen mixture (50% atmospheric humidity), initially at over 300° C. for 5 h and then at over 400° C. for 9 h.

### Examples 2 to 7 (Inventive)

The inventive VPO catalysts were produced in analogous manner to example 1, with the exception that the calcined powder of the catalyst precursor was mixed per 100 g with 4.5 g (example 2), 1.8 g (example 3), 5.0 g (example 4), 2.0 g (example 5), 1.0 g (example 6), 0.5 g (example 7) of ZnO powder (commercial ZnO, particle size<100 μm, impurity content less than 0.05% by weight) and 0.5 g (example 2) and 0.2 g (example 3) of MgO powder and at the same time with the graphite. This afforded calcined precursor powders containing 5.0% by weight of ZnO/MgO (example 2), 2.0% by weight of ZnO/MgO (example 3), 5.0% by weight of ZnO (example 4), 2.0% by weight of ZnO (example 5), 1.0% by weight of ZnO (example 6) and 0.5% by weight of ZnO (example 7).

The breaking strength of VPO catalysts according to examples 1 to 7 was in each case tested before and after activation. XRD diffractograms of the VPO catalysts were generated and these were tested in respect of catalytic selectivity.

The results presented in Table 1 and in FIG. 5 show that the addition of ZnO or ZnO/MgO after the calcining step results in no systematic increase in side crush strength (SCS) being observed in a measurement immediately after tableting. Surprisingly, this is however the case after the VPO catalyst tablets had undergone the subsequent activation step. Here, the particles with addition of ZnO or ZnO/MgO show a systematic and very clear increase in side crush strength. Table 1 and FIG. 5 also show that, under the chosen catalytic conditions (85% conversion), the addition of ZnO or ZnO/MgO leads to an increase in selectivity.

The VPO catalyst of the invention is additionally characterized by infrared spectroscopy features. Typical noninventive VPO catalysts without ZnO have a maximum in the IR spectrum at 790 cm⁻¹ to 810 cm⁻¹ (first absorption band) and a second maximum between 820 cm⁻¹ and 840 cm⁻¹ (second absorption band), where the second absorption band here is more intense than the first absorption band (FIG. 1, (1)). It has been found that the addition of ZnO or ZnO/MgO simultaneously causes an increase in the intensity of the band at 790 cm⁻¹ to 810 cm⁻¹ (first absorption band) and a decrease in the intensity of the maximum between 820 cm⁻¹ and 840 cm⁻¹ (second absorption band), such that the first absorption band in the samples of the invention is more intense than the intensity of the second absorption band, or the intensity of the second absorption band is even lowered to such an extent that it is no longer detectable (FIG. 1, (2)-(7)). In the latter case, it is a feature of the VPO catalysts of the invention that they have only the first absorption band.

It can also be seen that the VPO catalysts according to the invention have the following characteristics that are observable by XRD diffractometry (FIG. 6):
31.7 to 31.9° (sharp reflection, ZnO)
34.3 to 34.5° (sharp reflection, ZnO)
36.2 to 36.4° (sharp reflection, ZnO)

These characteristics become more intense with increasing ZnO content and are in good agreement with ZnO (in wurtzite structure/as zincite).

### Comparative Example 8, Examples 9, 10

To examine the correlation between the presence of the stabilizing ZnO phase and the time of addition of the binder, XRD diffractograms were recorded after different times of addition. Whereas in examples 2 to 7 ZnO was always added after calcining and before tabletting, the ZnO was added at the time of preparation of the reaction mixture during process step a), i.e. together with the addition of the V₂O₅ (comparative example 8), immediately after the filtration according to process step c) (example 9), or during process step d), i.e. after vacuum drying and before calcining (example 10). In these examples, 5.2 g of ZnO was in each case added, which means that the finished catalysts nominally contain in each case 2% by weight of ZnO.

FIG. 7 shows that the XRD reflections observed when ZnO is added after calcining and before tabletting, in particular those at 31.7 to 31.9°, 34.3 to 34.5°, 36.2 to 36.4°, are not visible when the ZnO is added at the start of the reflux step. (FIG. 7, diffractogram (8)).

### Comparative Examples 11 and 12

The VPO catalysts according to comparative examples 11 and 12 were produced in analogous manner to the VPO catalyst according to example 1, but with the activated tablets impregnated with Zn(OAc)₂ solution. Two different concentrations were used here so as to obtain a Zn/V ratio respectively of 0.008 (=0.29% by weight of Zn based on the total catalyst weight) and 0.016 (corresponding to 0.58% by weight based on the total catalyst weight). After impregnation the catalysts were dried in air, first for 18 h at 100° C. and then for a further 20 min at 350° C.

FIG. 8 shows the XRD diffractograms of the VPO catalysts according to comparative examples 11 and 12 (diffractograms (11) and (12)) in a comparison with the VPO catalyst according to example 5 produced according to the invention (diffractogram (5)). It can be seen that addition of the ZnO compound to the activated tablets does not give rise to any reflections, in particular at 31.7 to 31.9°, 34.3 to 34.5°, 36.2 to 36.4°.

### Comparative Examples 13 and 14

The VPO catalyst according to comparative example 13 was produced in analogous manner to the VPO catalyst according to example 2, but with 10% by weight of binder of the Secar 71 type and 4% by weight of graphite added to the calcined powder.

The stability of the VPO catalyst can be determined very easily by measuring the stability in respect of the by-product water, since a significant proportion of water vapour originating from the oxidation reaction is present during the reaction, which can damage the catalyst. The stability in respect of the by-product water of the inventive VPO catalyst according to example 4 was therefore tested in comparison with a VPO catalyst according to comparative example 13 produced with conventional binder. For this, the selectivity of the untreated samples was tested, then both samples were impregnated with water and the selectivity test repeated (comparative example 14). It was found that impregnation with water does not result in the catalyst of the invention incurring any loss of selectivity within the tolerance of the measurement.

### Examples 15 and 16

The VPO catalysts according to examples 15 and 16 were produced in analogous manner to the VPO catalyst according to example 1, but without addition of ammonium dimolybdate, with the result that the VPO catalyst obtained was free of Mo.

In addition, 1% by weight (example 15) or 2% by weight (example 16) of ZnO, based on the total weight of the calcined powder, was added to the calcined powder after calcining and before tabletting. As shown by Table 1, an increase in the catalytic selectivity combined with an increase in side crush strength was achieved also in the case of the Mo-free VPO catalysts. The transmission IR spectra shown in FIG. 2 additionally show that, in the case of these two samples of the invention, the intensity of the band at 790 cm⁻¹ to 810 cm⁻¹ (first absorption band) is greater than the maximum between 820 cm⁻¹ and 840 cm⁻¹.

### Examples 17 and 18 (Comparative)

### Apparatus Used

The same apparatus was used as in example 1.

### Preparation of the Catalyst Precursor

First, the four-necked flask is charged with 150 g of V₂O₅. To this is added 300 mL of benzyl alcohol and 1200 mL of isobutanol. The suspension is inertized with N₂ and then heated under reflux for 10 h while stirring. After cooling to max. 40° C. while stirring, 7.51 g of ammonium dimolybdate, 6.60 g of iron(III) nitrate nonahydrate, 2.55 g of cerium(III) nitrate hexahydrate and 4.5 g of niobium ammonium oxalate are added. After adding 138 mL of phosphoric acid (85%) over a 15-minute period while stirring and inertizing with N₂, the suspension is heated under reflux for a further 24 h while stirring.

### Filtration:

After cooling the suspension containing the catalyst precursor product, the suspension is transferred from the four-necked flask to a filter funnel and the liquid is removed by suction. The damp filter cake is washed once with ethanol (100%) and the liquid in the filter funnel again removed by suction. The same procedure is then repeated with double-distilled water.

### Drying/Calcining:

The washed filter cake is transferred to the evaporator flask of a rotary evaporator. The filter cake is dried at 120° C. overnight under water-jet vacuum. The powder dried in this way is placed in a furnace in a suitable calcining pot and calcined in an N₂ atmosphere at temperatures of 200° C. to 300° C. for 9 h.

### Compacting/Tabletting:

Before compacting/tabletting, first 2.1% by weight of Zn₃(PO₄)₂ and then 4% by weight of graphite are added to the calcined pulverulent catalyst precursor product and this is mixed homogeneously using a drum hoop mixer. This powder is compacted into plates with a roller compactor at a compaction pressure of 190 bar, a gap width of 0.60 mm and a roller speed of 7 rpm and granulated through a 1 mm screen.

The granulate is pressed with a rotary tablet press into the desired tablet shape having the appropriate height, for example 5.6×5.6×2.3 mm, and side crush strength.

### Heat-Treatment

The following heat-treatment is carried out under controlled conditions in a retort installed in a programmable furnace. The calcined tablets are evenly filled into the retort and the retort is sealed tightly. The catalyst is then treated at 120° C. for 18 h in a stream of air.

The VPO catalyst according to example 18 was produced in analogous manner to the VPO catalyst according to example 1, but with 2.1% by weight of Zn₃(PO₄)₂ based on the total weight of the calcined powder added to the calcined powder after calcining and before tabletting.

As shown in Table 1, the noninventive samples according to examples 17 and 18 show much lower selectivity compared to the inventive samples. At the same time, in the transmission IR spectra in FIG. 3, it is observed that, in the case of these two noninventive samples, the intensity of the band at 790 cm⁻¹ to 810 cm⁻¹ (first absorption band) is not greater than the maximum between 820 cm⁻¹ and 840 cm⁻¹ (example 17) or both bands are not present (example 18).

### Results

TABLE 1

| | | Amount | Side crush | Side crush | | |
|---|---|---|---|---|---|---|
| | | of | strength | strength | | Selectivity |
| | | binder | before | after | IR, | 85% |
| | | [% by | activation | activation | XRD | (MA) |
| Example | Binder | wt.] | [N] | [N] | FIG. | [%] |
| 1 | — | 0.0 | 23.4 | 23.5 | 1, 6 | 64.5 |
| (comparative) | | | | | | |
| 2 | 9ZnO/ | 5.0 | 24.1 | 35.5 | 1, 6 | 61.1 |
| (inventive) | 1MgO* | | | | | |
| 3 | 9ZnO/ | 2.0 | 26.1 | 28.7 | 1, 6 | 67.8 |
| (inventive) | 1MgO* | | | | | |
| 4 | ZnO | 5.0 | 21.1 | 35.5 | 1, 6 | 58.5 |
| (inventive) | | | | | | |
| 5 | ZnO | 2.0 | 20.9 | 27.5 | 1, 6, | 68.2 |
| (inventive) | | | | | 7, 8 | |
| 6 | ZnO | 1.0 | 20.2 | 26.1 | 1 | 66.8 |
| (inventive) | | | | | | |
| 7 | ZnO | 0.5 | 22.8 | 24.6 | 1 | 66.8 |
| (inventive) | | | | | | |
| 8 | ZnO | 2.0 | 24.0 | 22.2 | 7 | — |
| (comparative) | | | | | | |
| 9 | ZnO | 2.0 | — | — | 7 | — |
| (inventive) | | | | | | |
| 10 | ZnO | 2.0 | — | — | 7 | — |
| (inventive) | | | | | | |
| 11 | Zn(OAc)₂ | 0.3 | — | — | 8 | 64.8 |
| (comparative) | | | | | | |
| 12 | Zn(OAc)₂ | 0.6 | — | — | 8 | 59.9 |
| (comparative) | | | | | | |
| 13 | Secar | 10 | — | — | — | 64.6 |
| (comparative) | | | | | | |
| 14 | Secar | 10 | — | — | — | 58.0 |
| (comparative) | | | | | | |
| 15 | ZnO | 1.0 | 17.6 | 23.3 | 2 | 66.9 |
| (inventive) | | | | | | |
| 16 | ZnO | 2.0 | 15.7 | 27.5 | 2 | — |
| (inventive) | | | | | | |
| 17 | Zn₃(PO₄)₂ | 2.1 | 19.3 | 13.9 | 3 | 65.3 |
| (comparative) | | | | | | |
| 18 | Zn₃(PO₄)₂ | 2.1 | 24.0 | 14.3 | 3 | 62.5 |
| (comparative) | | | | | | |

### Methods

### Infrared Spectroscopy (IR)

The catalysts were characterized by means of infrared spectroscopy (IR) in transmission. This involves determining, in a wavelength-dependent manner, the intensity of the infrared radiation from a source (I₀) after passing through a sample (I). Absorption of the IR radiation and hence excitation of vibration modes in the material examined result in attenuation of the infrared radiation in a wavelength-dependent manner. For the spectrum, the absorption calculated via log(I₀/I) is plotted against wavenumber.

The measurements were conducted on a Nicolet Nexus 470 FTIR spectrometer with a liquid nitrogen-cooled mercury-cadmium-tellurium (MCT) detector, an “IR Source Everglo” as IR source and an HeNe laser for frequency calibration. For sample production, tablets of the respective catalysts were finely crushed in a mortar and pestle and, by means of a manual hydraulic press, about 10 g of material was used to produce a thin disc of diameter 11 mm. This was mounted in a vacuum chamber with KBr windows and positioned in the beam path. Subsequently, the sample was pretreated at 200° C. for 2 h and a pressure of 10⁻⁴ mbar in order to remove adsorbed water. For the measurements, the vacuum chamber containing the sample was cooled down to 77 K by means of liquid nitrogen, and the spectrum was recorded at a pressure of 10⁻⁴ mbar with a step width of 2 cm⁻¹, an aperture of 72 and an optical velocity of 0.64.

### Determination of the IR Absorption Maximum

The intensity of the two absorption bands with one maximum each in the range between 790 cm⁻¹ and 810 cm⁻¹, and between 820 cm⁻¹ and 840 cm⁻¹, corresponds to the intensity of absorption in the region of the band maximum compared to the spectral baseline. For the determination of the intensity of the absorption in the region of the maximum, in each case, a first straight line was drawn as the linear baseline such that it makes tangential contact with the spectrum, i.e. the baseline of the spectrum, immediately (i.e. within the frequency range up to an adjacent absorption band) to the left and right beneath the band in question. Then a line is run vertically downward from the band maximum (S0), i.e. in the direction of constant wavelength and decreasing absorption, and the point of intersection of this line with the linear baseline (S1) is determined. The intensity of the absorption band is defined as the distance from S0 to S1.

### Test of Breaking Strength

For the measurement of the breaking strength of the shaped bodies, a Zwick Z0.5 tester was used to determine the force needed for breakage. The measurements were carried out in accordance with the standard ASTM D4179. To dry the shaped bodies prior to the measurement, these were stored for at least 3 h at 100° C. in a drying oven and the subsequent measurement of the breaking strength carried out within max. 1 h after the end of drying. The tester was operated in accordance with the standard ASTM D4179 at a constant force rate of 20.0 N/s. For each example, 100 tablets were each positioned individually one after the other with the cylinder axis parallel to the measuring jaw surface (in standard ASTM D4179 the RADIAL CRUSH) and measured. From the 100 individual values, the average value was then determined of the force necessary in each case for breakage of the shaped body, which corresponds to the average breaking force of the shaped body. All data on side crush strength in this application refer to side crush strengths obtained by the method described herein.

### Powder X-Ray Diffraction (XRD)

The catalysts were characterized by X-ray powder diffraction (XRD). In this technique, X-rays undergo diffraction in the crystalline regions of the sample at various diffraction angles. The diffraction angle is measured/plotted in 2θ. Characteristic reflections occur as a function of the diffraction angle, depending on the phase present. On the basis of these diffraction patterns, an assignment of the diffraction pattern to the phases present can be made with the aid of a database.

The measurements were carried out on a D4 Endeavor from Bruker AXS with Cu-Kα radiation and a LYNXEYE detector. The diffractograms were recorded in a 2θ angle range of 5 to 50° with a step width of 0.02° in a recording time of 1.5 s per step with a fixed divergence slit of 0.3°. For the measurement, the samples were finely ground and compressed in a sample holder. The apparatus allowed the diffraction angle to be varied by tilting the sample. All information on XRD reflections in this application refers to XRD reflections obtained with this methodology.

For better comparability of the diffractograms, these were in each case normalized. For this, the data point having the lowest intensity value is first identified in each diffractogram. This value is subtracted from all intensity values of the respective diffractogram. The maximum intensity of the (024) reflection (present at a 2θ value of 28.4-28.5°) in the respective diffractogram is then determined. All intensity values of the respective diffractogram are divided by this value.

### Catalytic Test Reaction

To determine catalyst performance, after the preparation of the catalyst was complete (reflux, filtration, vacuum drying, calcining, compacting, tabletting, activation), the catalytic properties of all catalysts were tested in a bench-scale reactor with 1.5 mol % butane in air in a diluted catalyst bed (1:9 mixture of catalyst to inert ceramic rings). The selectivity in respect of maleic anhydride (MA) was determined at a reaction temperature of 410° C. (salt bath) via variation in the mass GHSV within a range from 1300 to 5500 l/kg/h by interpolation at a conversion of 85%.

1-22. (canceled)

23. A VPO catalyst for the oxidation of hydrocarbons with molecular oxygen, the VPO catalyst comprising vanadium, phosphorus and oxygen, wherein the VPO catalyst further comprises in the range of 0.05-7.0 wt.-% zinc based on the total weight of the VPO catalyst, the zinc being partly present as ZnO, the VPO catalyst having in transmission infrared spectroscopy has a first absorption band having a maximum between 790 cm⁻¹ and 810 cm⁻¹ and optionally a second absorption band having a maximum between 820 cm⁻¹ and 840 cm⁻¹, wherein only the first absorption band is present or the intensity of the first absorption band is greater than the intensity of the second absorption band, the VPO catalyst being in the form of a shaped body.

24. A VPO catalyst for the oxidation of hydrocarbons with molecular oxygen, wherein the VPO catalyst contains between 0.05% by weight and 7.0% by weight of zinc, the zinc being is partly present as ZnO, the VPO catalyst being in the form of a shaped body.

25. The VPO catalyst according to claim 23, wherein the intensity of each of the absorption bands is determined by drawing a first straight line such that it makes tangential contact with the spectrum immediately to the left and right beneath the band, then running a second straight line proceeding from the maximum of the band in the direction of constant wavelength and decreasing absorption and determining the point of intersection of the two straight lines, where the intensity of the absorption band is defined as the length between maximum and point of intersection.

26. The VPO catalyst according to claim 23, wherein a ratio between the intensity of the first absorption band and the intensity of the second absorption band is more than 1.1.

27. The VPO catalyst according to claim 23, wherein a ratio between the intensity of the first absorption band and the intensity of the second absorption band is more than 2.

28. The VPO catalyst according claim 23, having a Mo content of between 0.1% by weight and 1% by weight, based on the total weight of the VPO catalyst.

29. The VPO catalyst according to claim 23, having between 0.2% by weight and 4% by weight of zinc, based on the total weight of the VPO catalyst.

30. The VPO catalyst according to claim 23, having between 0.5% by weight and 3.5% by weight of zinc, based on the total weight of the VPO catalyst.

31. The VPO catalyst according to claim 23, comprising:
0.5% by weight to 7% by weight of Zn,
0% by weight to 0.7% by weight of Mo,
26% by weight to 31% by weight of V,
17% by weight to 21% by weight of P, and
3% by weight to 5% by weight of C,
in each case based on the total weight of the VPO catalyst.

32. The VPO catalyst according to claim 23, wherein the shaped body has a side crush strength of more than 25 N, the side crush strength being determined with a Zwick Z0.5 tester using the standard ASTM D4179 at a constant force rate of 20.0 N/s, wherein 100 tablets are each positioned individually and measured with the cylinder axis parallel to the measuring jaw surface so as to determine an average breaking force, which represents the side crush strength.

33. The VPO catalyst according to claim 23, wherein the VPO catalyst exhibits a powder X-ray diffraction pattern having reflections at 31.7 to 31.9°, 34.3 to 34.5°, and 36.2 to 36.4° in a measurement using a D4 Endeavor from Bruker AXS with Cu-Kα radiation and a LYNXEYE detector, the diffractograms having been recorded in a 26 angle range of 5° to 50° with a step width of 0.02° in a recording time of 1.5 s per step with a fixed divergence slit of 0.30.

34. The VPO catalyst according to claim 23, wherein the shaped body has a double-alpha shape with a height of 3 mm to 8 mm, a length of 5 mm to 9 mm, a width of 4 mm to 8 mm and an internal hole diameter of 1 mm to 4 mm.

35. The VPO catalyst according to claim 23, wherein the shaped body has a cylindrical shape with a height of 3 mm to 8 mm, an essentially round base surface with a diameter of 3 mm to 8 mm and a central axial opening with a diameter of 1 mm to 3 mm.

36. A process for producing a VPO catalyst according to claim 23, comprising the steps of:
preparing a catalyst precursor containing vanadyl hydrogen phosphate, and
shaping the catalyst precursor to give shaped bodies,
characterized in that ZnO is mixed with the catalyst precursor after step a).

37. A process according to claim 36, wherein the preparation of the catalyst precursor containing vanadyl hydrogen phosphate in step a) is carried out by reducing a V(V) compound in the presence of a P(V) compound with an organic reductant during a reflux step at standard pressure in an organic solvent.

38. The process according to claim 36, wherein the catalyst precursor obtained in step a) is filtered in a step a₁) and is dried and/or calcined in a step a₂) at a temperature of not more than 300° C.

39. The process of claim 38, wherein step a₂) takes place in two steps, wherein, in a first step, drying is carried out under reduced pressure at a temperature within a range from 90° C. to 140° C., for a period of from 1 h to 24 h and, in a second step, calcining is carried out in nitrogen at a temperature within a range from 230° C. to 290° C., for a period of from 1 h to 24 h.

40. The process of claim 36, further comprising activating the catalyst material in a gas mixture comprising air, inert gas and water vapour, at a temperature within a range from 300° C. to 500° C., for a period of from 1 h to 24 h.

41. A process for producing maleic anhydride by catalytic oxidation of n-butane, wherein a reactant gas comprising oxygen and n-butane is passed through a reactor containing a bed of the VPO catalysts according to claim 23.

42. The process of claim 41, wherein the bed of the VPO catalysts is present in the reactor at a temperature of between 300° C. and 420° C., and the reactant gas contains between 0.2 to 10% by volume of n-butane and between 5 and 50% by volume of oxygen and is passed through the reactor tube at a space velocity of 1100 h⁻¹ to 2500 h⁻¹.
